(19) United States
(12) Patent Application Publication
(10) Pub. No.: US 2004/0038922 A1
(43) Pub. Date: Feb. 26, 2004

(54) Vaccine composition

(76) Inventors: Jean Haensler, Pollionnay (FR); Christian Marcel Hurpin, St Didier au Mont d'Or (FR)

Correspondence Address: G. Kenneth Smith, Intellectual Property, Knerr Building, Route 611, Discovery Drive, Swiftwater, PA, 18370, US

(21) Appl. No.: 10/398,361

(22) PCT Filed: Oct. 8, 2001
(86) PCT No.: PCT/FR01/03098

(30) Foreign Application Priority Data
Oct. 6, 2000 (FR) 00/12808

(51) Int. Cl. A61K 48/00; A61K 39/21

(52) U.S. Cl. 514/44; 424/188.1

(57) ABSTRACT

The invention concerns a vaccine composition comprising at least an antigen, a cationic lipid and an immunostimulatory oligonucleotide. Said vaccine composition is particularly designed to induce an immune response of the Th1 type and a cytotoxic T response when administered by parenteral delivery, and to induce a Th2 type immune response when delivered through the mucous system. Said composition is of particular interest when the cationic lipid is DC chol.

The invention relates to the field of immunization compositions. More particularly, the invention relates to an adjuvanted immunization composition.

In the prior art, many adjuvants are known which can be used in the field of vaccines in order to improve the immune response induced when they are administered. Thus, for example, patent application WO 96/14831 describes the use of adjuvants consisting of amphipathic compounds comprising a lipophilic group derived from a sterol linked to a cationic group, such as 3β-[N-(N′,N′-dimethylaminoethane)carbamoyl] cholesterol, also called DC-chol.

Patent application WO 98/18810, itself, describes nucleotides, the nucleotide sequence of which has specific motifs (a CG dinucleotide framed by adenine, guanine or thymine on one side and cytosine or thymine on the other side), for their use as immunostimulants, in particular during the administration of vaccines.

These applications are merely examples among the considerable literature relating to this subject.

Now, although many substances have been described in the prior art regarding their immunization adjuvant properties, attempts are still being made to improve the quality and effectiveness of vaccines through, in particular, the use of novel adjuvants which would make it possible either to decrease the amount of antigens present in the vaccine in order to obtain a satisfactory immune response, or to orient the immune response in the desired direction as a function, for example, of the disease concerned, of the route of administration chosen or of the desired effect (prevention or treatment).

One of the difficulties is linked to the fact that, even though the responses of the immune system are increasingly well known, it remains very difficult, or even impossible, to anticipate them, and that, very often, the combination of 2 adjuvants produces a disappointing result, either because the toxicity is then too great or because each of the adjuvants, active individually, appears to have an inhibitory or neutralizing effect on the adjuvant which is combined with it.

The aim of the present invention is therefore to provide a novel immunization composition with an immunogenicity which is improved with respect to the prior art, i.e. the immune response induced consecutive to its administration is increased with respect to the prior art.

In order to achieve this aim, a subject-matter of the invention is an immunization composition comprising at least one antigen, one cationic lipid and one immunostimulant oligonucleotide.

Specifically, it has been noted, unexpectedly, that the adjuvant action of these 2 substances (the cationic lipid and the immunostimulant oligonucleotide) with respect to an antigen is synergistic when they are administered simultaneously.

A subject-matter of the invention is also the use of a composition comprising at least one cationic lipid and one oligonucleotide, for manufacturing a vaccine capable of inducing a Th1-type specific immune response when this composition is administered parenterally.

A subject-matter of the invention is also the use of a composition comprising at least one cationic lipid and one oligonucleotide, for manufacturing a vaccine capable of inducing a strong cytotoxic response, in particular a cytotoxic T response, when this composition is administered parenterally.

A subject-matter of the invention is also the use of a composition comprising at least one cationic lipid and one oligonucleotide, for manufacturing a vaccine capable of inducing a Th2-type specific immune response when this composition is administered mucosally.

A subject-matter of the invention is also the use of a composition comprising at least one antigen, one cationic lipid and one oligonucleotide, for manufacturing a vaccine capable of inducing a high production of IgA antibodies specific for said antigen, when this composition is administered mucosally.

According to one characteristic of the invention, said cationic lipid is DC-chol.

According to a specific characteristic of the invention, said antigen is an influenza virus antigen or an HIV virus antigen.

The present invention will be more clearly understood upon reading the detailed description which follows.

For the purpose of the present invention, the term “immunization composition” is intended to mean a composition which can be administered to humans or to animals in order to induce a response of the immune system, this response of the immune system possibly resulting in a production of antibodies or merely in activation of certain cells, in particular antigen-presenting cells, T lymphocytes and B lymphocytes. The immunization composition can be a composition for prophylactic purposes or for therapeutic purposes, or both.

The immunization composition can be administered via all the routes conventionally used in immunization; however, it has specific characteristics depending on the route of administration, in that it induces distinct specific immune responses. This is particularly advantageous if the intention is to direct the immune response against a particular antigen.

For example, in the case of microorganisms having a mucosal portal of entry, it may be advantageous to induce an immune response of mucosal type, with production of specific immunoglobulin A.

Thus, it may be advantageous to seek this type of response in immunization against viruses with a respiratory portal of entry (respiratory syncytial virus, influenza virus, parainfluenza virus, etc.), with a digestive portal of entry (poliovirus, rotavirus, etc.) or with a vaginal or rectal portal of entry (HIV, hepatitis B, etc.).

Similarly, an immune response of mucosal type is sought in bacterial ailments caused, for example, by Chlamydia, Neisseria gonorrheae, Streptococcus pneumoniae, Haemophilus influenzae or Moraxella catarrhalis.

On the other hand, in other cases, the intention is rather to induce a Th1-type response with production of cytotoxic cells; this is in particular the case for non-cytopathic viruses, such as cytomegaloviruses, intracellular microorganisms (Koch's bacillus, parasites such as Falciparum or Leishmania, bacteria such as Listeria, Legionella, Yersinia enterolitica) or other microorganisms, such as Spirochetes.

In certain cases, the induction of several types of response may be desired; this is in particular the case for influenza or Aids. In such cases, the composition according to the invention is of most particular value since it then makes it possible to produce various types of response of the immune system.

For the purpose of the present invention, the term “antigen” is intended to mean any antigen which can be used in a vaccine, whether it is a whole microorganism or a subunit, and whatever its nature: peptide, protein, glycoprotein, polysaccharide, glycolipid, lipopeptide, etc. They may be viral antigens, bacterial antigens or other antigens; the term “antigen” also comprises the polynucleotides for which the sequences are chosen so as to encode the antigens whose expression, by the individuals to which the polynucleotides are administered, is desired, in the case of the immunization technique called DNA immunization. It can also be a set of antigens, in particular in the case of a multivalent immunization composition which comprises antigens capable of protecting against several diseases, or in the case of a composition which comprises several different antigens in order to protect against a single disease, as is the case for certain vaccines against whooping cough or influenza, for example.

For the purpose of the present invention, the term “cationic lipid” is intended to mean a compound made up of a fatty portion (for example one or more hydrophobic chains or a sterol core) and of a polar head positively charged at physiological pH. In particular, it can be a compound comprising a lipophilic group derived from a sterol linked to a cationic group, and in particular a cholesterol derivative linked to a quaternary ammonium or to an amine which can be protonated via a carbamoyl linkage. Such a linkage in fact has the advantage of being hydrolyzable in the cell. Such compounds can be in basic form, in the form of a salt, or, and this is most commonly the case, in both forms in equilibrium in a mixture, the displacement of the equilibrium toward one or other form depending on the composition of the mixture and, in particular, on its pH. One of the cationic lipids which is particularly advantageous for the purposes of the invention is DC-chol, which can be produced from cholesteryl chloroformate and N,N-dimethylethylenediamine, according to the method described in U.S. Pat. No. 5,283,185 or, preferably, according to the method described in Example 8 of patent application WO 96/40067. It is also possible to use a product produced by reacting cholesteryl chloroformate and N,N,N-trimethylethylenediamine.

For the purpose of the present invention, the term “oligonucleotide” is understood to mean a single-stranded oligonucleotide having from 6 to 100 nucleotides, preferably from 6 to 30 nucleotides. It can be an oligoribonucleotide or an oligodeoxyribonucleotide. Use is in particular made of oligonucleotides comprising at least one Cytosine, Guanine dinucleotide sequence in which neither the Cytosine nor the Guanine is methylated. Any other oligonucleotide known to be, by its very nature, immunostimulant may also be suitable for the purposes of the invention. Particularly good results have been obtained using an oligonucleotide the sequence of which is described in patent application WO 96/02555 under SEQ ID No. 15, which is repeated hereinafter: 5′ GAGAACGCTCGACCTTCGAT 3′.

The oligonucleotides suitable for the purposes of the invention can be in the form of a phosphodiester or in any other form studied in order to improve them, in particular in terms of stability; thus, it is possible to use oligonucleotides which are in the form of phosphorothioates or of phosphodiester/phosphorothioate hybrids. Although it is possible to use oligonucleotides originating from existing nucleic acid sources, such as genomic DNA or cDNA, synthetic oligonucleotides are preferably used. Thus, it is possible to develop oligonucleotides on a solid support, using the β-cyanoethyl phosphoramidite method (Beaucage, S. L. and Caruthers, M. H. Tetrahedron Letters 22, 1859-1862 (1981)) for the 3′-5′ assembly.

In the phosphorothioated oligonucleotides, one of the oxygen atoms making up the phosphate group is replaced with a sulfur atom. The synthesis thereof can be carried out as described above, except that the iodine/water/pyridine tetrahydrofuran solution which is used during the oxidation step required for synthesizing the phosphodiester linkages is replaced with a TETD (tetraethylthiuram disulfide) solution to supply the sulfate ions allowing the production of the phosphorothioate group.

It is also possible to envisage other modifications of the phosphodiester linkages, of the bases or of the sugars, so as to modify the properties of the oligonucleotides used, and in particular so as to increase their stability.

For the purpose of the present invention, the expression “Th1-type immune response” is intended to mean an immune response specific for the antigen, characterized in that it causes directed production of cytokines, mainly γ-Interferon and IL2, and massive production of certain antibody subclasses (i.e. IgG2a in mice).

Production of cytotoxic T cells may also be observed.

The expression “Th2-type immune response” is intended to mean an immune response which results in production mainly of IL4 and IL5, and also in massive production of certain other antibody subclasses (i.e. IgG1 in mice).

When the intention is to study the type of immune response induced by an immunization composition, comparative assays of the specific IgG1s and IgG2as produced when the immunization composition studied is administered to mice can be carried out; a Th1-type response results in a greater production of specific IgG2as, producing a low value for the IgG1/IgG2a ratio, while a Th2-type response results in a greater production of specific IgG1s, producing a high value for the IgG1/IgG2a ratio.

Alternatively, assaying the cytokines produced also makes it possible, in in vitro assays or on animals, to assess the direction of the immune response; in particular the IL5/γINF ratio can be calculated; a Th1-type response results in a low value for this ratio, whereas a Th2-type response results rather in a high value for this ratio.

It is also possible to observe the amount of IgA, the production of which reflects an immune response directed toward the Th2 type.

Now, depending on the immunization targets, i.e. the diseases against which the immunization compositions are intended to be, it may be desirable to be able to direct the immune response.

The examples which follow illustrate, in a nonlimiting way, embodiments of the invention.

### EXAMPLE 1

DC-Chol hydrochloride (obtained according to the preparation method described in Example 8 of patent application WO 96/40067) was used, which was suspended at 20 mg/ml in TRIS-NaCl buffer (20 mM TRIS, 150 mM NaCl, pH 6.8). After 8 hours with stirring at 35 to 40° C. in an argon stream, the suspension was microfluidized using an M-110S microfluidizer from Microfluidics (10 cycles at 500 kPa), in order to generate a homogeneous suspension of DC-chol, which was filtered through a Millex 0.45 μm filter.

### EXAMPLE 2

Oligonucleotides were prepared using an automatic synthesizer machine supplied by Applied Biosystems, which uses the standard chemical phosphoramidite method and which includes an oxidation step in each cycle.

This oxidation step was carried out using an iodine/water/tetrahydrofuran/acetonitrile solution to obtain a phosphodiester linkage, and using a tetraethylthiuram/acetonitrile solution to obtain a phosphorothioate linkage.

An oligonucleotide 3 Db(S) was thus prepared, the sequence of which is reproduced in patent application WO 96/02555 under SEQ ID NO 15, and which includes phosphorothioate linkages throughout its length.

An oligonucleotide MGC (S) was also prepared, the sequence of which is reproduced in patent application WO 00/15256 in SEQ ID NO 2, which includes both phosphodiester linkages and phosphorothioate linkages. The phosphorothioate linkages are located at each end; there are 2 phosphorothioate linkages in 3′ and 5 phosphorothioate linkages in 5′. This oligonucleotide has no CG sequence and is used as a negative control.

### EXAMPLE 3

0.2 ml doses of immunization compositions against influenza were prepared, having one of the following formulations:

monovalent influenza vaccine strain A/Singapore/6/86 (H1N1) corresponding to 5 μg of HA alone,

monovalent influenza vaccine strain A/Singapore/6/86 (H1N1) corresponding to 5 μg of HA+200 μg of DC-chol prepared in Example 1,

monovalent influenza vaccine strain A/Singapore/6/86 (H1N1) corresponding to 5 μg of HA+50 μg of oligonucleotide 3Db(S) prepared in Example 2,

monovalent influenza vaccine strain A/Singapore/6/86 (H1N1) corresponding to 5 μg of HA+200 μg of DC-chol prepared in Example 1+50 μg of oligonucleotide 3Db(S) prepared in Example 2.

The doses prepared were administered to 4 groups of 6 Balb/c mice by peritoneal injection, as a 1^{st} injection on D0 and a booster injection on D21.

On D35, blood samples were taken from each mouse in order to assay the antibodies produced, using the ELISA technique. The assay results obtained are shown in Table 1 hereinafter, in which the titers given are means of the titers obtained by ELISA on each of the 6 mice belonging to each group.

| | IgG1 | IgG2a | IgG1/IgG2a |
|---|---|---|---|
| 5 μg HA | 20401 | 4930 | 9.1 |
| 5 μg HA + 200 μg DC- | 127743 | 10082 | 12.7 |
| chol | | | |
| 5 μg HA + 50 μg | 27243 | 15863 | 1.7 |
| 3Db (S) | | | |
| 5 μg HA + 200 μg DC- | 122956 | 87761 | 1.4 |
| chol + 50 μg 3Db (S) | | | |

These results illustrate the synergy obtained between the 2 adjuvants present in the immunization composition according to the invention, with regard to the production of IgG2a antibodies. Specifically, the amount of IgG2a antibodies produced after administration of an immunization composition according to the invention is clearly greater than the sum of the amounts produced after administration of the immunization compositions comprising just one of the adjuvants of the prior art.

In order to study the cytotoxic response induced, the spleen cells of the mice of each of the groups were removed on D35.

The cells, regarding which the intention was to measure the specific cytotoxic activity against target cells exhibiting a dominant class I MHC-restricted hemagglutinin epitope, were restimulated in vitro in the presence of syngeneic stimulating cells (derived from nonimmunized mice) infected with the A/Singapore/6/86 (H1N1) strain virus.

Their cytotoxic function was demonstrated using, as target cells, cells of the P815 line sensitized with a hemagglutinin epitope peptide of the A/Singapore/6/86 (H1N1) strain virus.

Target-cell lysis was measured using a radioactive technique based on loading the target cells with radioactive chromium Cr-51, and on the release of this radioelement during cell lysis.

For each of the immunization compositions assayed, the cytotoxic cells were brought into contact with the target cells in the following proportions: 100 cytotoxic cells per target cell, and 33 cytotoxic cells per target cell.

For each 100 or 33 value of the cytotoxic cell/target cell ratio, the following was carried out:

the chromium released spontaneously without adding cytotoxic cells was assayed,

the chromium released after total lysis of the target cells was assayed,

and also the chromium released after the action of the cells for which the intention is to measure the cytotoxic activity was assayed.

Then, the percentage of cytotoxicity was calculated in the following way:

$$\frac{100 \times ( cytotoxic cell release - spontaneous release )}{( total release - spontaneous release )}$$

The results obtained are given in Table 2 below:

TABLE 2

| | 100/1 | 30/1 |
|---|---|---|
| 5 μg HA | 43 | 28 |
| 5 μg HA + 200 μg DC-chol | 25 | 17 |
| 5 μg HA + 50 μg 3Db (S) | 49 | 19 |
| 5 μg HA + 200 μg DC-chol + | 71 | 46 |
| 50 μg 3Db (S) | | |

These results show that the cellular response assessed through cytotoxic cell induction is also increased when an immunization composition according to the invention is used.

The results obtained in a similar assay with nonsensitized target cells produce the following results given in Table 3 hereinafter:

TABLE 3

| | 100/1 | 30/1 |
|---|---|---|
| 5 μg HA | 7 | 4 |
| 5 μg HA + 200 μg DC-chol | 7 | 4 |
| 5 μg HA + 50 μg 3Db (S) | 10 | 9 |
| 5 μg HA + 200 μg DC-chol + | 8 | 4 |
| 50 μg 3Db (S) | | |

These results indicate that the cytotoxic response induced is a CD8+ cytotoxic T-cell response.

If all of the results obtained are considered, it is noted that the subject of the present invention makes it possible to direct the specific-antibody response toward a Th1-type immune response with a very substantial decrease in the IgG1/IgG2a ratio, while at the same time maintaining the level of specific IgG1 production equivalent to that obtained when the immunization composition comprises only one adjuvant consisting of DC-chol. This direction of the antibody response is also advantageously combined with induction of cytotoxic cells, and in particular of CD8+ T cells.

### EXAMPLE 4

0.2 ml doses of immunization compositions against influenza were prepared as in Example 3, having one of the following formulations:

monovalent influenza vaccine strain A/Singapore/6/86 (H1N1) corresponding to 5 μg of HA alone,

monovalent influenza vaccine strain A/Singapore/6/86 (H1N1) corresponding to 5 μg of HA+200 μg of DC-chol prepared in Example 1,

monovalent influenza vaccine strain A/Singapore/6/86 (H1N1) corresponding to 5 μg of HA+5 μg of oligonucleotide 3Db(S) prepared in Example 2,

monovalent influenza vaccine strain A/Singapore/6/86 (H1N1) corresponding to 5 μg of HA+200 μg of DC-chol prepared in Example 1+5 μg of oligonucleotide 3Db(S) prepared in Example 2.

Mice divided up into 4 groups of 6 were injected, subcutaneously this time, with a dose of each of the immunization compositions (1 group of 6 mice per immunization formulation) on D0 and on D21. The sera were sampled and assayed in the same way as in the previous experiment. The results obtained relating to the assays of the antibodies produced are given in Table 4 hereinafter:

| | IgG1 | IgG2a | IgG1/IgG2a |
|---|---|---|---|
| 5 μg HA | 6882 | 585 | 11.8 |
| 5 μg HA + 200 μg DC- | 108211 | 20443 | 5.3 |
| chol | | | |
| 5 μg HA + 50 μg | 4519 | 384 | 11.8 |
| 3Db (S) | | | |
| 5 μg HA + 200 μg DC- | 133544 | 59545 | 2.2 |
| chol + 50 μg 3Db (S) | | | |

The cytotoxicity assays showed, in the same way as in Example 3, that, with a composition according to the invention, cytotoxic cells, and in particular CD8+ T cells, were induced.

In addition, γ-Interferon assays showed that there was considerable induction of the production of this cytokine.

These results show, in the same way as in Example 3, that there is synergy between the effect of the 2 adjuvants, in particular regarding the IgG2a response, even when the amount of oligonucleotide is decreased to a value at which its adjuvant effect was not detectable. Unlike that which is observed conventionally, it is also noted that there is no inhibitory effect of one of the adjuvants on the action of the other when a composition according to the invention is used.

### EXAMPLE 5

Immunization compositions against the type 1 human immunodeficiency virus (HIV-1) were prepared, in which the antigen is the gp160 MN/LAI-2 envelope glycoprotein. This antigen contains the gp120 portion of the HIV-1 MN isolate and the gp41 portion of the HIV-1 LAI isolate. The gp41 has been deleted of its site of cleavage with the gp120 and of its transmembrane portion, so as to obtain a noncleaved and essentially secreted glycoprotein. The antigen is produced using the BHK-21 hamster cell line infected with the recombinant vaccinia virus VVTG.9150 derived from the preceding construct VVTG.1163 (Kieny, M. -P. et al., 1988, Protein Eng, 2(3): 219-255), and is then purified by ion exchange chromatography followed by immunoaffinity chromatography.

The 20 μl immunizing doses corresponded to one of the following formulations:

25 μg of gp160 only,

25 μg of gp160+50 μg of oligonucleotide 3Db(S) prepared in Example 2,

25 μg of gp160+50 μg of oligonucleotide MGC prepared in Example 2+200 μg of DC-chol prepared in Example 1,

25 μg of gp160+50 μg of oligonucleotide 3Db(S) prepared in Example 2+200 μg of DC-chol prepared in Example 1.

Four groups of 6 mice were injected with the immunizing doses prepared (1 formulation per group), rectally, under anesthetic, as 4 injections each separated by 2 weeks (namely D1, D15, D29 and D44).

On D57, a sample of serum was taken, the feces were recovered and rectal washes were performed in order to carry out the following assays:

assay of the anti-gp 160 IgGs in the serum, by ELISA,

assay of the total IgAs and IgGs, and also of the specific anti-gp160 IgAs and IgGs in the rectal washes, by ELISA,

assay of the total IgAs and IgGs and also of the specific anti-gp160 IgAs and IgGs in the feces, by ELISA.

The immunization composition containing the oligonucleotide MGC was considered to be a negative control with respect to oligonucleotide 3Db(S). Specifically, the oligonucleotide MGC had proved not to be immunostimulant in previous experiments.

The results obtained are shown in the tables hereinafter; only the means per group of mice having received the same immunization composition are indicated.

TABLE 5
Assay of specific IgGs in the serum:

| | Anti-gp 160 IgG in μg/ml |
|---|---|
| 25 μg gp160 | 60.55 |
| 25 μg gp160 + 50 μg 3Db (S) | 46.97 |
| 25 μg gp160 + 200 μg DC-chol + | 47.85 |
| 50 μg MGC | |
| 25 μg gp160 + 200 μg DC-chol + | 645.26 |
| 50 μg 3Db (S) | |

These results show the synergy exerted by the 2 adjuvants for the production of IgG against the gp160 antigen, when administration is via the mucous membrane route.

TABLE 6
Assay of the IgAs and of the IgGs in the
rectal washes:

| | Spec. | Spec. |
|---|---|---|
| | IgA/tot. IgA | IgG/tot. IgG |
| | in % | In % |
| 25 μg gp160 | 0.15 | 3.68 |
| 25 μg gp160 + 50 μg 3Db (S) | 0.91 | 2.46 |
| 25 μg gp160 + 200 μg DC- | 0.68 | 1.07 |
| chol + 50 μg MGC | | |
| 25 μg gp160 + 200 μg DC- | 1.53 | 12.43 |
| chol + 50 μg 3Db (S) | | |

TABLE 7
Assay of IgAs and of IgGs in the faeces:

| | Spec. | Spec. |
|---|---|---|
| | IgA/tot. IgA × | IgG/tot. IgG |
| | 10⁴ | in % |
| 25 μg gp160 | 2.44 | 0.00 |
| 25 μgp160 + 50 μg 3Db (S) | 18.05 | 1.33 |
| 25 μg gp160 + 200 μg DC- | 43.38 | 0.00 |
| chol + 50 μg MGC | | |
| 25 μg gp160 + 200 μg DC- | 104.79 | 3.03 |
| chol + 50 μg 3Db (S) | | |

These results show the synergistic effect obtained using the subject of the present invention, with respect to the local production of specific immunoglobulin G and specific immunoglobulin A.

This capacity to locally stimulate the production of specific IgAs is particularly desired in certain immunization applications, and confirms the value of the subject-matter of the present invention.

1. Immunization composition comprising at least one antigen, one cationic lipid and one immunostimulant oligonucleotide.

2. Immunization composition according to claim 1, characterized in that said cationic lipid is DC-chol.

3. Immunization composition according to one of the preceding claims, characterized in that said antigen is an influenza virus antigen.

4. Immunization composition according to one of claims 1 to 2, characterized in that said antigen is an HIV virus antigen.

5. Immunization composition according to one of the preceding claims, characterized in that it is intended for mucous membrane administration.

6. Immunization composition according to one of claims 1 to 4, characterized in that it is intended for parenteral administration.

7. Use of a composition comprising at least one antigen, one cationic lipid and one oligonucleotide, for manufacturing a vaccine capable of inducing a Th1-type immune response when it is administered parenterally.

8. Use of a composition comprising at least one antigen, one cationic lipid and one oligonucleotide, for manufacturing a vaccine capable of inducing a high production of IgA antibodies specific for said antigen, when it is administered mucosally.

9. Use of a composition comprising at least one antigen, one cationic lipid and one oligonucleotide, for manufacturing a vaccine capable of inducing a cytotoxic T immune response when it is administered parenterally.

10. Use of a composition comprising at least one antigen, one cationic lipid and one oligonucleotide, for manufacturing a vaccine capable of inducing a Th2-type immune response when it is administered mucosally.

11. Use according to one of claims 7 to 10, characterized in that said cationic lipid is DC-chol.
